(19) United States
(12) Patent Application Publication
(10) Pub. No.: US 2025/0259728 A1
(43) Pub. Date: Aug. 14, 2025

(54) SYSTEMS, DEVICES, AND METHODS FOR MONITORING, ANALYZING, AND/OR PROVIDING FEEDBACK REGARDING USE OF A THERAPEUTIC RESPIRATORY DEVICE

(71) Applicant: Airotone, Inc., Naples, FL (US)

(72) Inventor: Gregory O'KEEFFE, Naples, FL (US)

(21) Appl. No.: 18/856,482

(22) PCT Filed: Apr. 14, 2023
(86) PCT No.: PCT/US2023/018736

§ 371 Date: Oct. 11, 2024

Related U.S. Application Data
(60) Provisional application No. 63/331,208, filed on Apr. 14, 2022.

(51) Int. Cl. G16H 20/30; A61B 7/00

(52) U.S. Cl. CPC G16H 20/30; A61B 7/003; A61B 2560/0462; A61B 2562/0204; A61B 2562/0219

(57) ABSTRACT

Respiratory therapy sound capturing devices may be configured to cooperate with respiratory therapy devices such as an inhaler, a positive expiratory pressure (PEP) device, oscillating positive expiratory pressure (oPEP) device, a peak flow meter. Incentive spirometer, spirometer, and a respiratory muscle trainer (RMT) device to record sounds associated with use of the respiratory therapy device. The respiratory therapy sound capturing devices may include an attachment mechanism configured to attach the respiratory therapy sound capturing device to a respiratory therapy device and a microphone configured to detect sound made when a user is using the respiratory therapy device and communicate the detected sound to a transceiver communicatively coupled to the microphone and transmit sound detected by the microphone to an external computing device.

### RELATED APPLICATION

This application is a patent application filed under the Patent Cooperation Treaty and claims priority to U.S. Provisional Patent Application No. 63/331,208, which was filed on 14 Apr. 2022 and entitled “SYSTEMS, DEVICES, AND METHODS FOR MONITORING, ANALYZING, AND/OR PROVIDING FEEDBACK REGARDING USE OF A THERAPEUTIC RESPIRATORY DEVICE” and is incorporated, in its entirety, herein.

### FIELD

The present invention is directed to medical devices and methods of use thereof. In particular, the systems, devices, and methods for monitoring, analyzing, and/or providing feedback regarding use of a respiratory therapy device.

### BACKGROUND

Performing breathing exercises and receiving feedback as to a volume of air inhaled or exhaled and analyzed to determine the lung capacity or respiratory and/or lung function of a user typically requires the use of cumbersome and sometimes expensive equipment. Proper and regular use of traditional respiratory therapy and/or diagnostic equipment is usually limited to clinical settings where patient engagement can be ensured under direct oversight. Once discharged, only a fraction of patients continues with their breathing exercises as instructed, exposing non-compliant patients to respiratory complications such as pneumonia.

### SUMMARY

Exemplary respiratory therapy sound capturing devices disclosed herein may include an attachment mechanism (e.g., an adhesive) configured to attach the respiratory therapy sound capturing device to a respiratory therapy device, a microphone configured to detect sound made when a user is using the respiratory therapy device and communicate the detected sound to a transceiver communicatively coupled to the microphone and configured to transmit sound detected by the microphone to an external computing device. The respiratory therapy device may be, for example, a medication inhaler, an incentive spirometer, a positive expiratory pressure (PEP) device, a respiratory muscle trainer (RMT), a spirometer, a nebulizer, an oscillating positive expiratory pressure (oPEP) device, and/or a peak flow meter. The sound detected by the microphone may be, for example, sound corresponding to at least one of air moving into the respiratory therapy device, air moving out of the respiratory therapy device, air moving through a respiratory therapy device, a vibration of the respiratory therapy device, and/or an oscillation of a component air of the respiratory therapy device.

On some occasions, the respiratory therapy sound capturing device also include an accelerometer configured to detect at least one of motion of the respiratory therapy sound capturing device and an orientation of the respiratory therapy sound capturing device.

In some embodiments, the respiratory therapy sound capturing device may include a mouthpiece and a body and, in these embodiments, the respiratory therapy sound capturing device may be configured to be placed between the mouthpiece and the body of the respiratory therapy device to capture sound the user makes when inhaling out of the mouthpiece or exhaling into the mouthpiece. At times, the respiratory therapy sound capturing device may further comprises a lumen configured to allow air exhaled into mouthpiece to be communicated to the body of the respiratory therapy device and is also configured to allow air inhaled through the body of the respiratory therapy device to be communicated to the mouthpiece.

Exemplary system disclosed herein may include a respiratory therapy device and a respiratory therapy sound capturing device coupled to the respiratory therapy device, the respiratory therapy sound capturing device comprising a microphone configured to detect sounds made when a user is using the respiratory therapy device, and communicate the detected sound to a transceiver communicatively coupled to the microphone and configured to transmit sound detected by the microphone to an external computing device.

Exemplary methods that may executed by a processor, or computing device, communicatively coupled to, for example, a respiratory therapy sound capturing device includes receiving a parameter for using a respiratory therapy device, receiving sound detected by a microphone when a user is using the respiratory therapy device and communicating the detected sound to a transceiver, and evaluating and/or analyzing, by the processor, the received sound to determine if the received sound corresponds to use of the respiratory therapy device in a manner that is compliant with the received parameter. Then, a result of the analysis may be provided to a display device. In some embodiments, the analysis may include determining, for example, a number of inhales in a time period, a number of exhales in a time period, a duration of inhales in a time period, a duration of exhales in a time period. Additionally, or alternatively, the analysis may include determining an average frequency, a peak frequency, an average amplitude, a peak amplitude and/or a duration detected for one or more exhalations detected during a time period, an average frequency, a peak frequency, an average amplitude, a peak amplitude, and/or a duration detected for one or more inhalations detected during a time period. In some embodiments, the method may further include determining a recommendation regarding how to use the respiratory therapy device responsively to the analysis and providing the recommendation to the user.

### BRIEF DESCRIPTION OF THE DRAWINGS

The present invention is illustrated by way of example, and not limitation, in the figures of the accompanying drawings in which:

FIG. 1A is a block diagram of an exemplary respiratory therapy sound capturing device, consistent with some embodiments of the present invention;

FIG. 1B is a cross-section diagram of an exemplary respiratory therapy sound capturing device, consistent with some embodiments of the present invention;

FIG. 1C is a cross-section diagram of an exemplary respiratory therapy sound capturing device that includes a diaphragm, consistent with some embodiments of the present invention;

FIG. 1D1 is a diagram of a side view of an exemplary respiratory therapy sound capturing device, consistent with some embodiments of the present invention;

FIG. 1D2 is a diagram of a front view respiratory therapy sound capturing device of FIG. 1D1, consistent with some embodiments of the present invention;

FIG. 1E1 is a diagram of a side view of a mouthpiece that includes a respiratory therapy sound capturing device for use with a respiratory therapy device, consistent with some embodiments of the present invention;

FIG. 1E2 is a diagram of a front view of the mouthpiece of FIG. 1E1, consistent with some embodiments of the present invention;

FIG. 1F1 is a diagram of a side view of a respiratory mask for use with a respiratory therapy device that incorporates a respiratory therapy sound capturing device, consistent with some embodiments of the present invention;

FIG. 1F2 is a diagram of a front view of the respiratory mask of FIG. 1F1, consistent with some embodiments of the present invention;

FIG. 1G1 is a diagram of a side view of an exemplary mouthpiece that includes a sound-producing component and a respiratory therapy sound capturing device, consistent with some embodiments of the present invention;

FIG. 1G2 is a diagram of a vertical cross section of the mouthpiece of FIG. 1G1 bisected along line A-A, consistent with some embodiments of the present invention;

FIG. 1G3 is a diagram of a vertical cross section of the mouthpiece of FIG. 1G1 bisected along its length, consistent with some embodiments of the present invention;

FIG. 2A is a block diagram of an exemplary respiratory therapy sound capturing device coupled to an exemplary respiratory therapy device, consistent with some embodiments of the present invention;

FIG. 2B is diagram of a side view of an exemplary system that includes a respiratory therapy device and a respiratory therapy sound capturing device affixed thereto, consistent with some embodiments of the present invention;

FIG. 2C is a front view of the respiratory therapy device of FIG. 2B, consistent with some embodiments of the present invention;

FIG. 2D is a back view of mouthpiece of the respiratory therapy device of FIG. 2B, consistent with some embodiments of the present invention;

FIG. 2E is a side view of an exemplary system that includes a respiratory therapy device with a respiratory therapy sound capturing device positioned between mouthpiece and a body 220 of the respiratory therapy device, consistent with some embodiments of the present invention;

FIG. 2F is a side view of an exemplary system that includes the mouthpiece of FIG. 2E1 coupled to the respiratory therapy device of FIG. 2B, consistent with some embodiments of the present invention;

FIG. 2G is a side view of an exemplary system that includes the mouthpiece shown in FIG. 1G1 coupled to a respiratory therapy device of FIG. 2B, consistent with some embodiments of the present invention;

FIG. 2H is a side view of an exemplary system that includes respiratory mask shown in FIG. 1F1 coupled to a respiratory therapy device of FIG. 2B, consistent with some embodiments of the present invention;

FIG. 2I is a diagram of an assembly of the respiratory therapy device of FIG. 2B, a secondary respiratory therapy device, and the respiratory therapy sound capturing device of FIG. 1D1, consistent with some embodiments of the present invention;

FIG. 2J provides a diagram of an assembly of the respiratory therapy device of FIG. 2B, a secondary respiratory therapy device, and the respiratory therapy sound capturing device of FIG. 1E1, consistent with some embodiments of the present invention;

FIG. 2K provides a diagram of an assembly of the respiratory therapy device of FIG. 2B, a secondary respiratory therapy device, and the mouthpiece of FIG. 1G1, consistent with some embodiments of the present invention;

FIG. 2L provides a diagram of an assembly of the respiratory therapy device of FIG. 2B, a secondary respiratory therapy device, and the respiratory mask of FIG. 1F1, consistent with some embodiments of the present invention;

FIG. 3 is a block diagram of an exemplary system configured to execute one or more methods disclosed herein, consistent with some embodiments of the present invention;

FIG. 4A is a flowchart illustrating a process for using a respiratory therapy sound capturing device to measure and/or record sound associated with use of a respiratory therapy device and/or provide feedback to a user of the respiratory therapy sound capturing device regarding his or her use of the respiratory therapy device, consistent with some embodiments of the present invention;

FIG. 4B is a flowchart illustrating a process for evaluating a sample of a recording of sound associated with use of a respiratory therapy device, consistent with some embodiments of the present invention;

FIG. 5 provides a spectrogram showing sound recorded by a respiratory therapy sound capturing device, consistent with some embodiments of the present invention;

FIG. 6A provides a graph showing observed sound volume, or intensity, as a function of air flow rate measured for a particular user inhaling from a system including a respiratory therapy device that is coupled to one or more of the respiratory therapy sound capturing device(s), consistent with some embodiments of the present invention;

FIG. 6B provides a graph showing observed sound volume, or intensity, as a function of air flow rate measured for a particular user exhaling into a system including a respiratory therapy device that is coupled to one or more of the respiratory therapy sound capturing device(s), consistent with some embodiments of the present invention;

FIG. 7A provides a graph showing a first relationship between sound amplitude as a function of frequency when a user is using a first respiratory therapy device, consistent with some embodiments of the present invention;

FIG. 7B provides a graph showing the first relationship of FIG. 7A overlaid upon a second relationship between sound amplitude as a function of frequency when a user is using a second respiratory therapy device, consistent with some embodiments of the present invention;

FIG. 7C provides a graph showing the first relationship of FIG. 7A overlaid upon a third relationship between sound amplitude as a function of frequency when a user is using a third respiratory therapy device, consistent with some embodiments of the present invention;

FIG. 7D provides a graph showing the first relationship of FIG. 7A (when the user is using the first respiratory therapy device at a first setting (e.g., level of resistance)) overlaid upon a fourth relationship between sound amplitude as a function of frequency when a user is using the first respiratory therapy device but with a different setting, consistent with some embodiments of the present invention; and

FIG. 7E provides a graph showing the first relationship of FIG. 7A overlaid upon a fifth relationship between sound amplitude as a function of frequency when a user is shaking the respiratory therapy device and/or respiratory therapy sound capturing device, consistent with some embodiments of the present invention.

Throughout the drawings, the same reference numerals, and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### Written Description

Proper use of a respiratory or other medication dispensing device (e.g., an inhaler) and/or a respiratory (inhalation and/or exhalation) training, diagnostic, and/or therapeutic device such as a positive expiratory pressure (PEP) device and/or oscillating positive expiratory pressure (oPEP) device, medication inhalers, respiratory muscle trainers (RMT), incentive spirometers, peak flow meters, and other spirometry devices (which may be collectively referred to herein as “respiratory therapy devices”) substantially contributes to improved respiratory diagnosis and/or outcomes when performing respiratory therapy and respiratory training. Without direct observation of a patient's use of a respiratory therapy device, it is difficult, if not impossible, for respiratory therapists and other treatment providers to ascertain whether a patient is employing proper technique when he or she is using a respiratory therapy device. This greatly impairs the treatment provider's ability to monitor use of the respiratory therapy device and provide feedback regarding, for example, unobserved (e.g., at-home) usage of a respiratory therapy device and/or compliance with a treatment plan and/or plan of care. The systems, devices, and methods disclosed herein are designed and programmed to observe, measure, and/or record characteristics of a patient's use of a respiratory therapy device that may be used to evaluate a patient's use of the respiratory therapy device and/or evaluate whether a technique of use is correct and/or aligned with proper technique for using the particular respiratory therapy device. The evaluation of measured, recorded, and/or observed characteristics of a patient's use of a respiratory therapy device may be done by, for example, an on-board processor, a software application running on a user device (e.g., smart phone or computer), and/or by a processor or server of, for example, a treatment provider and/or third party operating via, for example, the Internet and/or cloud computer processing and/or data storage. A result of this evaluation may be, for example, a measured value, an indexed value, or grade (e.g., 1-10 or A-F) indicating a degree of compliance with, for example, a treatment plan, a parameter of a respiratory training method or program, and/or a recommendation for improving use for the respiratory training device.

In some embodiments, the systems and devices disclosed herein may be configured to be removably attached to a respiratory therapy device so that, for example, the respiratory therapy device may be cleaned between uses and/or the respiratory therapy device may be coupled to a wired or wireless battery charging device and/or computing device such as a smart phone or computer. The systems and devices may be, for example, incorporated into a mouthpiece for use with a respiratory therapy device, configured for insertion into a breath pathway for the respiratory therapy device, and/or configured to be attached to an exterior or interior surface of the respiratory therapy device.

The devices and systems disclosed herein may be configured to receive, measure, and/or record sound produced by a respiratory therapy device during use by a patient or user and/or sounds generated by the patient/user (e.g., coughing, wheezing, etc.) before, during, and/or after use of the respiratory therapy device. Optionally, the devices and systems disclosed herein may also be configured to measure and/or record motion of the device during use.

Additionally, or alternatively, the devices and systems disclosed herein may be configured to measure and/or record other sounds made by the body such as heartbeat and/or breathing sounds. At times, these systems and devices may be configured to move, independently, or in cooperation with, another mechanism (e.g., a track or strap) to different locations on the body to, for example, find a position of the body (e.g., chest or back) that provides the clearest audio signal.

In some embodiments, the systems and devices disclosed herein may be configured to provide a user with feedback regarding how he or she is using a respiratory therapy device. This feedback may include, for example, indications of whether, or not, the user is breathing through the respiratory therapy device a prescribed number of times, with a prescribed amount of expiratory force, for a prescribed length of time for each breath, at a prescribed time of day, and/or whether the user is employing a proper technique (e.g., execution of a series of steps and/or compliance with one or more parameters for proper and/or prescribed use of the respiratory therapy device) for use of the respiratory therapy device. Additionally, or alternatively, the systems and devices disclosed herein may be configured to provide orientation information for the respiratory therapy device and the feedback to the user may include adjusting an orientation of the respiratory therapy device during use.

At times, this feedback may be provided to a treatment provider (e.g., a doctor or respiratory therapist) of the user so that the treatment provider may, for example, adjust a prescribed use of the respiratory therapy device, train the user to properly use the respiratory therapy device, improve the technique of using the respiratory therapy device, and/or change a type of respiratory therapy device used.

In some embodiments, the systems, devices, and methods disclosed herein may be configured and/or designed to analyze and/or record when a user is speaking to analyze the speech for breathing and/or other respiratory patterns such as how many breaths are taken over a period of time (e.g., 15 s, 30 s, or 60 s) and/or a duration of time the user pauses to take a breath while speaking. At times, the analyzed speech may be a set of words (e.g., two-ten-word sentences) that is repeated by the user when speech is being recorded/analyzed and/or when the systems and/or devices disclosed herein are used. Additionally, or alternatively, the analyzed speech may be recorded throughout the day at, for example, random or scheduled intervals and/or during participation in particular activities (e.g., exercising or sleeping). This recorded speech may then be analyzed to determine respiratory patterns of the user. The determined respiratory patterns may be used to adjust one or more parameters and/or prescribed usage of a respiratory therapy device.

On some occasions, the systems and devices disclosed herein may be configured to put a microphone in close proximity to a sound-producing device and/or a mouthpiece of a respiratory therapy device, which may enable setting a gain, or sound sensitivity, of the microphone to a relatively low value, which may reduce and/or eliminate an amount of ambient noise that is detected by the microphone while allowing for accurate capture of sounds made while using a respiratory therapy device.

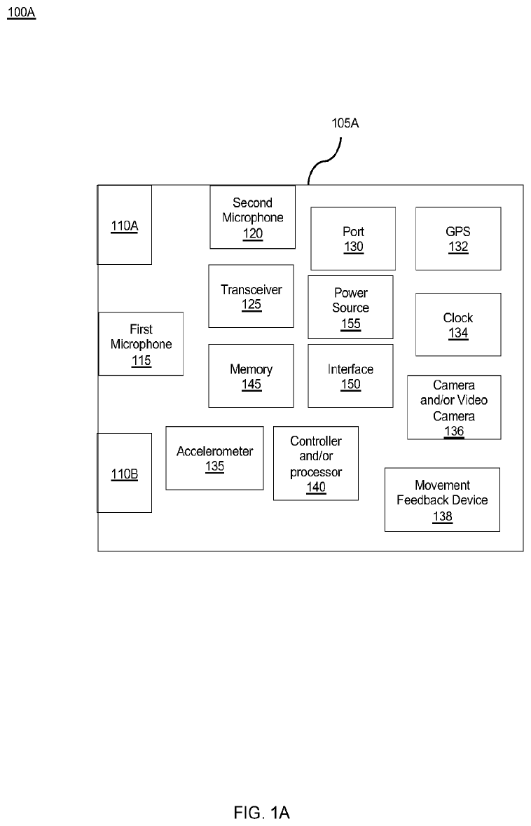

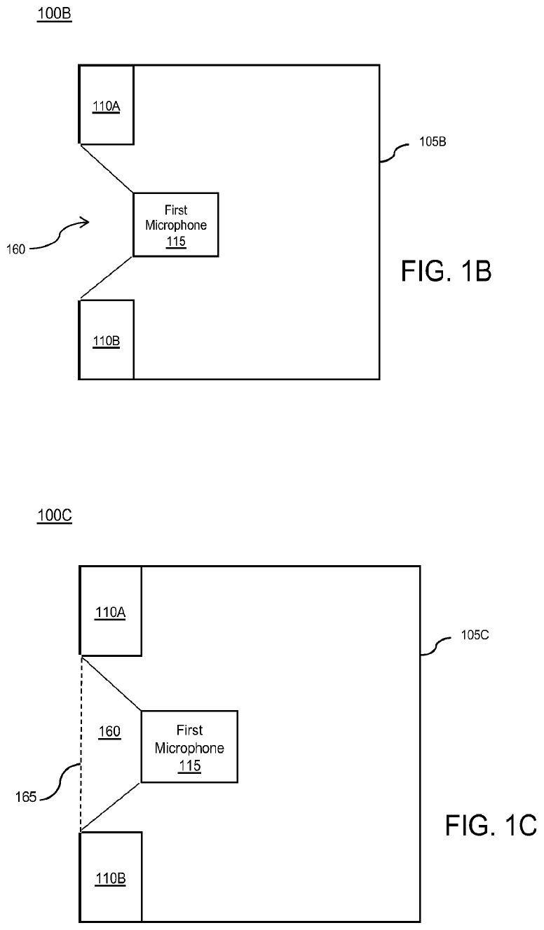

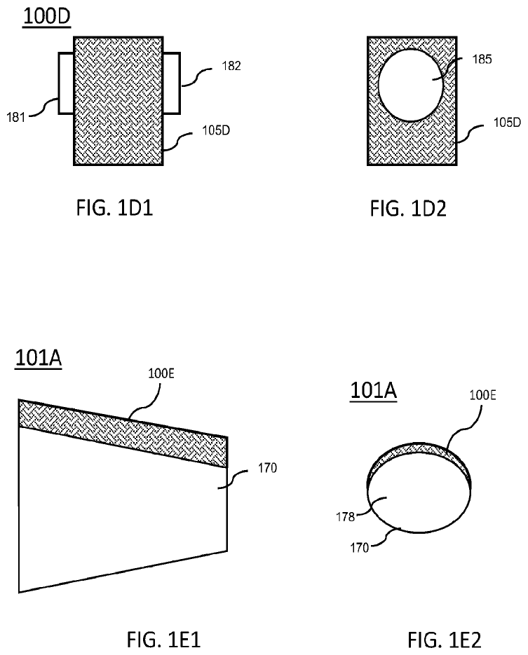

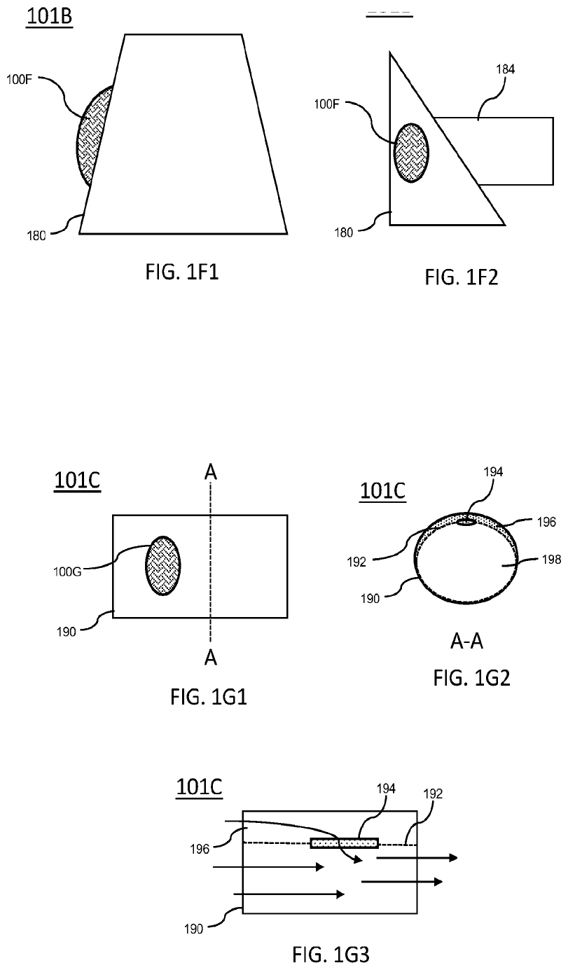

Turning now to the figures, FIG. 1A provides a block diagram of an exemplary respiratory therapy sound capturing device 100A that includes a first optional adhesive mechanism 110A, a second optional adhesive mechanism 110B, a first microphone 115, an optional second microphone 120, a transceiver 125, a port 130, a global positioning device (GPS) 132, a clock 134, an accelerometer 135, a camera and/or a video camera 136, a movement feedback device 138, a controller and/or processor 140, a memory 145, an interface 150, and a power source 155; all of which may be enclosed within a housing 105A. In some embodiments, respiratory therapy sound capturing device 100A may be waterproof by virtue of, for example, housing 105A.

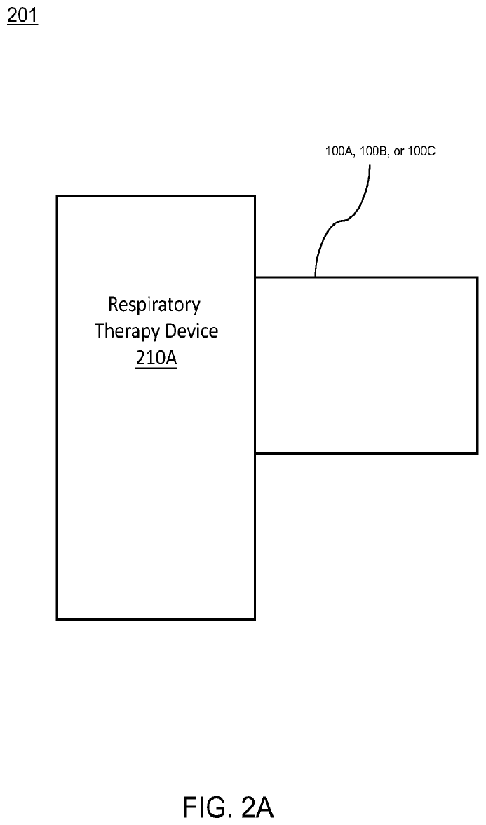

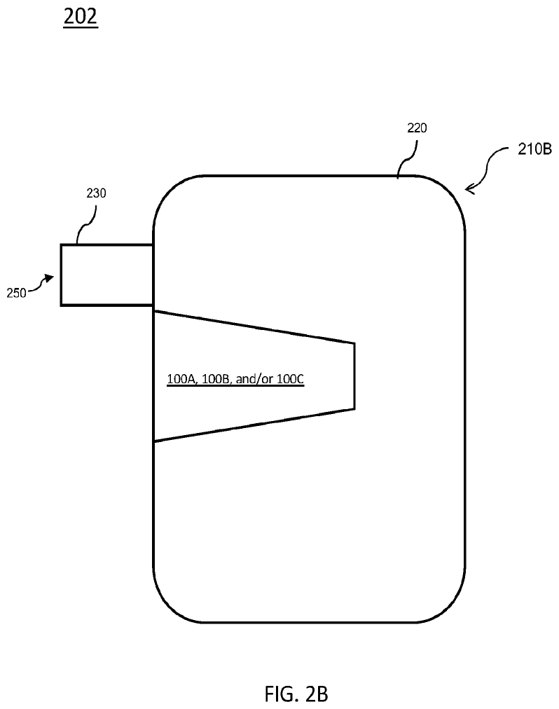

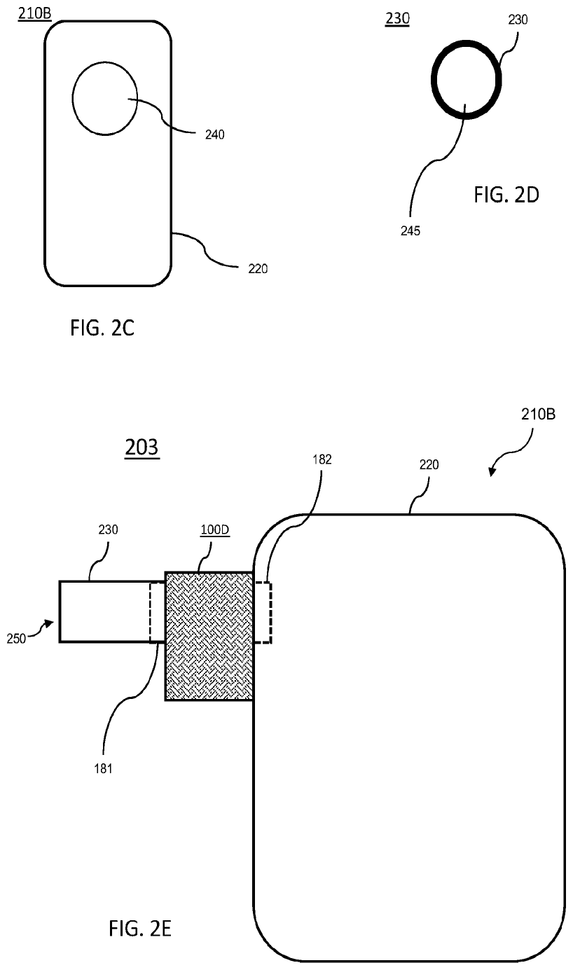

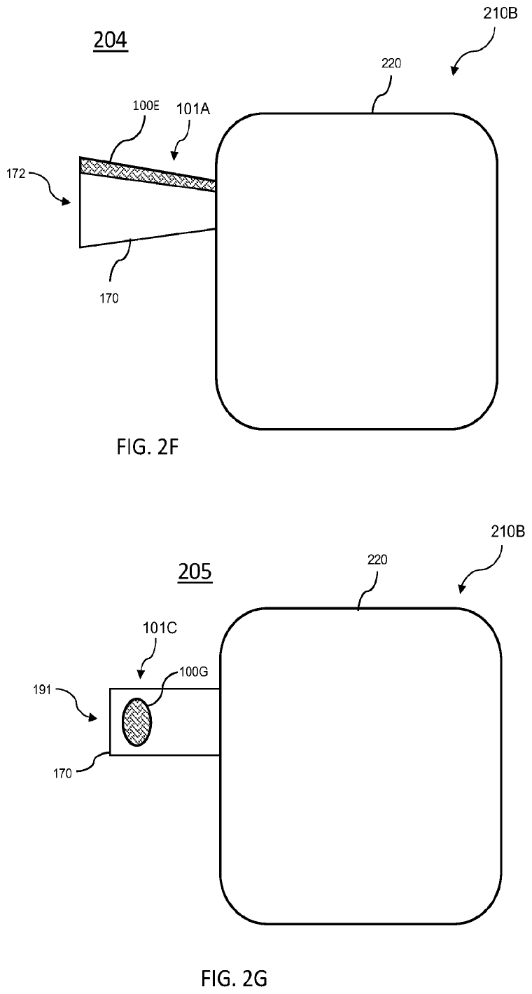

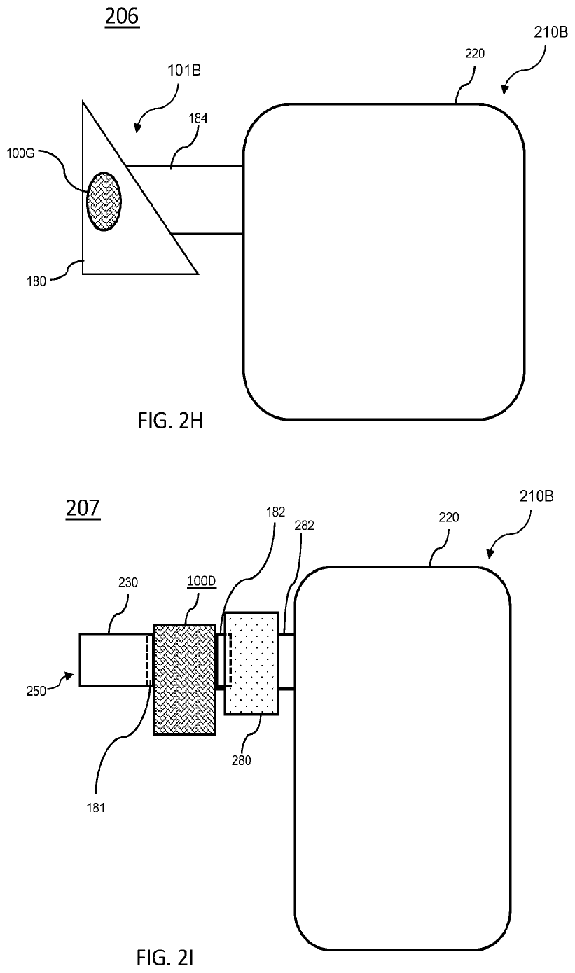

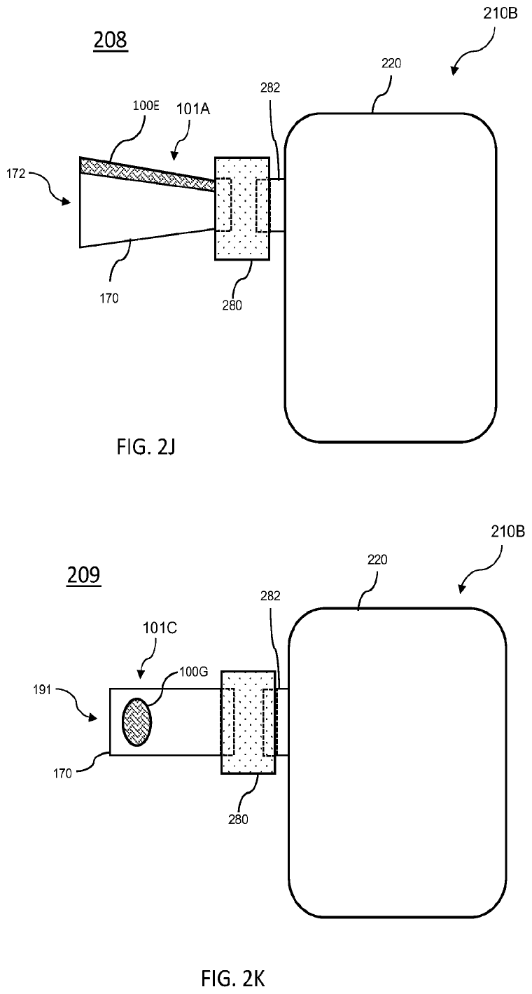

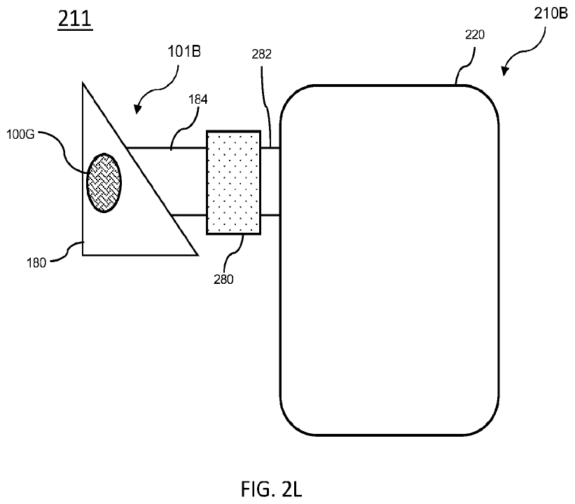

Housing 105A may be temporarily and/or permanently affixed to a respiratory therapy device (not shown) via, for example, optional first and/or second attachment mechanism(s) 110A and/or 110B. Exemplary optional first and/or second attachment mechanism(s) 110A and/or 110B include, but are not limited to, glue, adhesive, magnets, snaps, straps, VELCRO™, and/or clasp. In some embodiments, optional first and/or second attachment mechanism(s) 110A and/or 110B may be configured to attach to a corresponding attachment mechanism of a respiratory therapy device and/or wearable device such as a strap to be worn around the wrist or neck in a manner similar to a watch or necklace, respectively. Additionally, or alternatively, optional first and/or second attachment mechanism(s) 110A and/or 110B may be a coupling (e.g., male/female coupling, leur lock, twits lock, etc.) configured to couple with and/or integrate into one or more components of a respiratory therapy device, examples of which are shown in FIGS. 2A-2H and discussed herein.

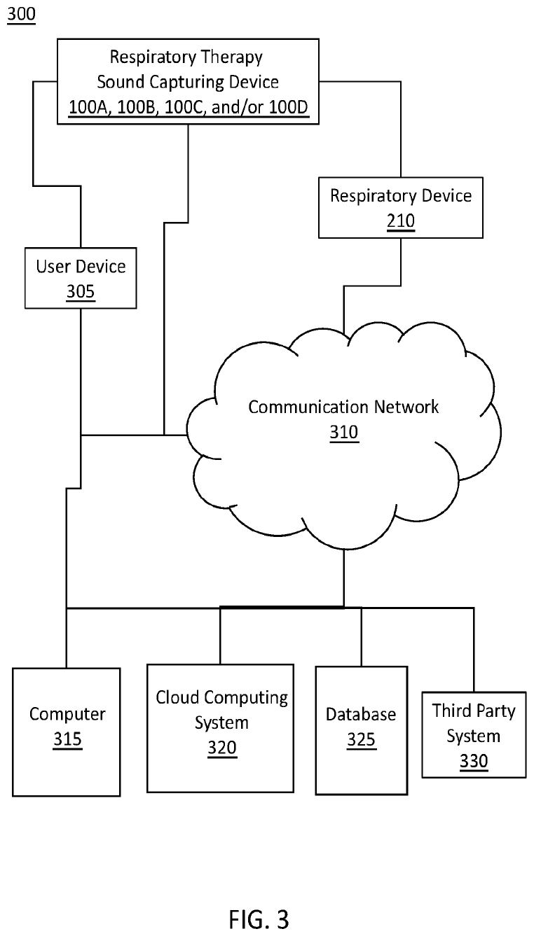

First microphone 115 may be arranged within housing 105A so that it may be proximate and/or affixed to (via, e.g., first and/or second adhesive mechanisms 110A and/or 110B) a surface of a respiratory therapy device (not shown) so that sound made by the respiratory therapy device (e.g., flutters and/or movement of components) during use by a patient may be received, measured, and/or recorded in memory 145. In some instances, first microphone 115 may be positioned within and/or proximate to a mechanical sound amplification feature of housing 105A such as a cone or parabolic depression in a portion of the housing configured to abut an exterior surface of the respiratory therapy device as shown in FIG. 1B, which is a cross-section diagram of an exemplary respiratory therapy sound capturing device 100B that includes a housing 105B that houses all the components of housing 105A and further includes a concave depression, or indentation, 160 with first microphone positioned at an approximate apex of indentation 160, and one or more components of respiratory therapy sound capturing device 100A (not shown). Indentation 160 may be shaped to focus and/or direct sound coming from, for example, a respiratory therapy device and/or a human (e.g., chest or back) to first microphone 115. Optional second microphone 120 may be configured to, for example, record sounds (e.g., coughs, wheezing, etc.) in an ambient environment and/or of a patient using the respiratory therapy device and/or detect ambient noise, which may then be used as, for example, an input for a noise cancelling algorithm. In some embodiments, first and/or second microphone(s) 115 and/or 120 may be, for example, a contact microphone or an omnidirectional microphone. Transceiver 125 may be configured to receive, for example, instructions for operation (e.g., on/off, communication of recordings and/or measurements, etc.) of respiratory therapy sound capturing device 100A and/or receipt of, for example, operational instructions. Additionally, or alternatively, transceiver 125 may be configured to transmit data and/or information received and/or processed by, for example, first microphone 115, second microphone 120, accelerometer 135, controller and/or processor 140, and/or interface 150 to, for example, a user device and/or external computer or system of computers as, for example, shown and discussed below with regard to FIG. 3. Transceiver 125 may operate via, for example, wired (e.g., via port 130) and/or wireless communication. Additionally, or alternatively, port 130 may be an electrical coupling to, for example, power source 165 that may be used to, for example, charge power source 165 which may be embodied as a rechargeable battery. Additionally, or alternatively, port 130 may be a wireless communication port and/or transceiver (e.g., transceiver 125) configured to communicate with one or more external computing devices via one or more wireless communication protocols such as BLUETOOTH™.

Accelerometer 135 may be configured to determine motion (e.g., acceleration) of respiratory therapy sound capturing device 100A and/or an orientation thereof. In some embodiments, accelerometer 135 may be and/or may include a gyroscope that may be configured to determine motion (e.g., acceleration) of respiratory therapy sound capturing device 100A and/or an orientation thereof. Additionally, or alternatively, accelerometer 135 may be configured to determine motion of the user when, for example, using respiratory therapy sound capturing device 100A and/or during waking hours.

Controller and/or processor 140 may be configured to control an operation of one or more components of respiratory therapy sound capturing device 100. User interface 150 may be configured to accept and/or communicate to the user one or more instructions (e.g., on/off, mode of operation, etc.) and may be and/or include a user interface such as, for example, a speaker, a button, a touch-sensitive display, a keypad, and/or combinations of same. Additionally, or alternatively, controller and/or processor 140 may be configured to receive a sound signal (e.g., a signal indicative of sound generated via using a respiratory therapy device and/or an respiratory therapy sound capturing device as disclosed herein) from first and/or second microphone 115 and/or 120, process and/or analyze the sound signal, and/or communicate the sound signal to transceiver 125 for communication to a device external to respiratory therapy sound capturing device 100A (e.g., a computer or smart device (e.g., phone, tablet, etc.) and/or one or more components of system 300 discussed below with regard to FIG. 3). The processing and/or analysis of the sound signal may include, for example, application of a noise reduction process or algorithm, filtering the sound to remove sound of unwanted frequencies or intensities, amplifying the sound, or a portion thereof, to amplify sound of wanted frequencies or intensities, determining whether an intensity and/or volume of the sound is within a specified range, and/or determining whether a sound measurement and/or recording is compliant with one or more instructions for the use of a respiratory therapy sound capturing device (e.g., recording is of a sufficient length). At times, activation (e.g., initiation of measurement and/or recording) of sound by respiratory therapy sound capturing device 100A may be responsive to detection of a sound by first microphone 115 and/or second microphone 120 and/or motion detected by, for example, and/or accelerometer 135 and, in this way, respiratory therapy sound capturing device 100A may be sound, noise, and/or motion activated. In some embodiments, this activation may be responsive to detection of a particular type and/or frequency of sound (e.g., sound frequencies known to be produced by a respiratory therapy device such as the respiratory therapy devices as shown and discussed herein) and/or vibratory patterns caused by, for example, an oscillating component and/or valve of a respiratory therapy device.

Clock 134 may be any timekeeping device and may be configured to time stamp one or more sounds, measurements of sound, and/or recordings of sound detected by respiratory therapy sound capturing device 100, first microphone 115 and/or second microphone 120. GPS device 132 may be configured to determine and/or triangulate a position for respiratory therapy sound capturing device 100A and/or cooperate with, for example, a user device (as described herein) to obtain a position for respiratory therapy sound capturing device 100A. On some occasions, GPS device 132 may be a SIM card. Camera and/or video camera 136 may be configured and/or arranged to enable it to photograph and/or record a video of the user using respiratory therapy sound capturing device 100A. These images and/or video recordings may be provided to, for example, a clinician, coach, and/or respiratory therapist via transceiver 125 and/or port 130 so that, for example, they may be analyzed to determine whether the user is using a respiratory therapy device with proper technique. Movement feedback device 138 may be any device configured to provide a user with feedback when he or she is moving respiratory therapy sound capturing device and/or a respiratory therapy device coupled thereto. In some instances, movement feedback device 138 may be a bead or other mechanical device that moves when respiratory therapy sound capturing device 100A moves and strikes a surface causing noise. In one example, movement feedback device 138 may be a bead that moves back and forth along a track when respiratory therapy sound capturing device 100A (or a device coupled thereto, such as a medication inhaler) is moved up and down.

FIG. 1C is a cross-section diagram of an exemplary respiratory therapy sound capturing device 100C that is configured as an auscultation device that includes depression 160, a housing 105C that houses, for example, one or more components of respiratory therapy sound capturing device 100A (not shown), and a diaphragm 165. Diaphragm 165 may be configured to amplify sound emanating from the user's body such as a heartbeat and/or a sound generated by the lungs while breathing. In some embodiments, diaphragm 165 may be removable from housing 105C via, for example, a clip, track, strap, and/or adhesive so that a user may add diaphragm 165 to housing 105C in advance of performing auscultation and remove diaphragm 165 from housing 105C following performance of auscultation and/or when use of respiratory therapy sound capturing device 100C to measure, record, and/or analyze sound made by the user when, for example, using respiratory therapy sound capturing device 100C to do respiratory training.

FIG. 1D1 provides a side view and FIG. 1D2 provides a front view of an exemplary respiratory therapy sound capturing device 100D that includes a housing 105E that houses, for example, one or more components of respiratory therapy sound capturing device 100A and includes a first end coupling 181, a second end coupling 182, and a lumen 185. First end coupling 181 may be sized, positioned, and/or configured so that an external diameter thereof may fit securely within an interior diameter of a mouthpiece for a respiratory therapy device such as the mouthpieces and/or respiratory therapy devices disclosed herein. Second end coupling 182 may be sized, positioned, and/or configured so that an external diameter thereof may fit securely within an interior diameter of a coupling, opening, or hole, in a respiratory therapy device otherwise configured to, for example, cooperate with and accept insertion of the mouthpiece. First and/or second end couplings 181 and/or 182 may be secured and/or held in place via, for example, friction, a gasket, a lock, and/or a screw on/off mechanism. In some embodiments, first end coupling 181 may be sized, positioned, and/or configured to have an internal diameter sized to fit over an exterior diameter of second end coupling 182.

FIG. 1E1 is a diagram of a side view and FIG. 1E2 is a diagram of a front view of a mouthpiece 101A for use with a respiratory therapy device that incorporates a respiratory therapy sound capturing device 100E that may include one or more components respiratory therapy sound capturing device 100A. In the embodiment shown in FIGS. 1E1 and 1E2, respiratory therapy sound capturing device 100E is incorporated into a housing positioned on top of (as oriented in FIGS. 1E1 and 1E2) a mouthpiece body 170, which may have a hole, or lumen, 178 through which a user may inhale air from a respiratory therapy device and/or exhale air into the respiratory therapy device. In the embodiment of FIGS. 1E1 and 1E2, first microphone 115 may be a contact microphone in contact with an outer diameter of mouthpiece body 170 proximate to lumen 178. Mouthpiece 101A may be configured and/or designed to replace a standard mouthpiece typically used with a respiratory therapy device.

FIG. 1F1 is a diagram of a side view and FIG. 1F2 is a diagram of a front view of a respiratory mask 101B for use with a respiratory therapy device that incorporates a respiratory therapy sound capturing device 100F that may include one or more components respiratory therapy sound capturing device 100A. In the embodiment shown in FIGS. 1F1 and 1F2, respiratory therapy sound capturing device 100F is incorporated into a housing affixed to an exterior surface of respiratory mask 101B. Respiratory mask 101B have a body 180 that is configured to fit over a user's nose and mouth, thereby capturing air inhaled into and/or exhaled from the patient's nose and/or mouth as it is respectively inhaled into and/or exhaled from a respiratory therapy device, which may be coupled to respiratory mask 101B via a coupling 184. Respiratory mask 101B may be configured and/or designed to replace a standard mouthpiece typically used with a respiratory therapy device.

FIG. 1G1 is a diagram of a side view of an exemplary mouthpiece 101C that has a body 190 and a respiratory therapy sound capturing device 100G affixed to an exterior surface of body 190. FIG. 1G2 is a diagram of a vertical cross section of body 190 that is perpendicular to a length of body 190 along line A-A and FIG. 1G3 is a diagram of a vertical cross section of body 190 that is parallel (as shown in FIG. 1G1) to a length of body 190. Mouthpiece 101C includes a sound-producing component 194 positioned along a membrane 192 that separates a first chamber 196 of mouthpiece 101C from a central lumen 198 of mouthpiece 101C. Sound-producing component 194 may be configured to make sound when air passes over and/or through it as shown in FIG. 1G3, wherein air exhaled by a user is shown with arrows, which show a direction of air movement from an entrance of mouthpiece 101C on the left toward the right (as oriented in the figure) and into a respiratory therapy device (not shown). Exemplary sound-producing devices include, but are not limited to, reeds, paper, and porous membranes. Mouthpiece 101C may be configured so that all air inhaled and/or exhaled by the user passes through mouthpiece 101C to/from respiratory therapy device. Stated differently, mouthpiece 101C may be configured so that air exhaled into chamber 196 passes through sound-producing device 194 and on to a respiratory therapy device coupled thereto with no loss or gain of inhaled and/or exhaled air to or from the environment. In this way, mouthpiece 101C may be configured so that it does not inhibit, or otherwise impact, an operation or function of the respiratory therapy device. FIG. 2A is a diagram of an exemplary system 201 that includes respiratory therapy sound capturing device(s) 100A, 100B, or 100C coupled via, for example, chemical, mechanical, and/or magnetic means, to an exemplary respiratory therapy device 210A that may be, for example, a device whose use is designed to provide one or more therapies to a patient/user. Exemplary respiratory therapy devices 210A include, but are not limited to, positive expiratory pressure (PEP) devices, airway clearance devices, positive expiratory pressure (OPEP) devices, medication inhalers, peak flow meters, spirometers, incentive spirometers, and respiratory muscle resistance training (RMT) devices.

FIG. 2B is a side view of an exemplary system 202 that includes a respiratory therapy device 210B with a body 220 and a mouthpiece, or tube, 230 through which a patient may inhale and/or exhale. FIG. 2B also shows respiratory therapy sound capturing device 100A, 100B, or 100C being affixed to an external surface of body 220 as shown. A front end 250 of mouthpiece 230 may be of any appropriate shape (e.g., circular, oval, etc.) that may be, for example, comfortable for the user to place his or her mouth over and exhale and/or inhale into. In some embodiments, respiratory therapy sound capturing device(s) 100A, 100B, and/or 100C may be configured to be in an air stream of an inhalation and/or exhalation that is directed into and/or out of respiratory therapy device 210B so that, for example, it does not interfere with an air pathway and/or all air inhaled and/or exhaled by the user is delivered to and/or extracted from respiratory therapy device 210A. In these embodiments, an assembly of respiratory therapy device 210A and respiratory therapy sound capturing device(s) 100A, 100B, and/or 100C may be a closed system in that air inhaled and/or exhaled by the user does not escape via respiratory therapy sound capturing device(s) 100A, 100B, and/or 100C.

FIG. 2C is a front view of respiratory therapy device 210B and shows an opening 240 in body 220 sized, positioned, and configured for cooperation with and acceptance of a corresponding outer diameter of mouthpiece 230 when mouthpiece and body 220 are assembled together. FIG. 2D is a back view of mouthpiece 230 and shows a central lumen 245 of mouthpiece 230 along with the circumferential shape of the back of mouthpiece 230, wherein an exterior diameter of the circumferentially shaped back of mouthpiece 230 is configured for insertion into and/or cooperation with opening 240 as shown in, for example, FIG. 2B. FIG. 2E is a side view of another exemplary system 203 that includes respiratory therapy device 210B with a respiratory therapy sound capturing device 100A, 100B, 100C, or 100D positioned between mouthpiece 230 and body 220 as shown, wherein first end coupling 181 is inserted into central lumen 245 of mouthpiece 230 and second end coupling 182 is inserted into opening 240 as shown. In this way, respiratory therapy sound capturing device 100A, 100B, 1000, or 100D may be positioned between mouthpiece 230 and/or body 220 and capture information about a patient's and/or user's use of breathing device 210B while the user/patient is using respiratory therapy device 210B to perform respiratory therapy.

FIG. 2F is a side view of an exemplary system that includes mouthpiece 101A coupled to respiratory therapy device 210B so that a user may exhale into and/or inhale from respiratory therapy device 210B via an opening 172 to lumen 178 (not shown) of mouthpiece 101A while respiratory therapy sound capturing device 100E detects, measures, and/or records sound while, for example, the user is using respiratory therapy device 210B as disclosed herein.

FIG. 2G is a side view of an exemplary system that includes mouthpiece 101C coupled to respiratory therapy device 210B so that a user may exhale into and/or inhale from respiratory therapy device 210B via an opening 191 to lumen 198 (not shown) of mouthpiece 101C while respiratory therapy sound capturing device 100G detects, measures, and/or records sound while, for example, the user is using respiratory therapy device 210B as disclosed herein.

FIG. 2H is a side view of an exemplary system that includes respiratory mask 101B coupled to respiratory therapy device 210B so that a user may exhale into and/or inhale from respiratory therapy device 210B while respiratory therapy sound capturing device 100G detects, measures, and/or records sound while, for example, the user is using respiratory therapy device 210B as disclosed herein.

In some embodiments, one or more of the respiratory therapy sound capturing device(s) 100A, 100B, and/or 100C, mouthpieces 101A and/or 101C, and/or respiratory mask 101B may be assembled with a primary respiratory therapy device, such as respiratory therapy device 210B, and a second respiratory therapy device, such as the respiratory therapy devices disclosed herein, a nebulizer, a source of medication, a source of oxygen, and/or source of steam. These assemblies may be coupled together using any appropriate means including, but not limited to, couplings, tube extensions, clips, gaskets, and clamps. FIGS. 2I-2L provides some examples of such assemblies, wherein FIG. 2I is a diagram of an assembly 207 of respiratory therapy device 210B, which is coupled to a secondary respiratory therapy device 280 via a coupling 282 configured to fit within the interior diameter of opening 240 and couple secondary respiratory therapy device 280 to respiratory therapy device 210B. Assembly 207 also includes respiratory therapy sound capturing device 100D, which is coupled to secondary respiratory therapy device 280 and mouthpiece 230 in a manner similar to that discussed above with regard to FIG. 2E.

FIG. 2J provides a diagram of an assembly 208 of respiratory therapy device 210B, which is coupled to a secondary respiratory therapy device 280 via coupling 282, which extends into secondary respiratory therapy device 280, thereby coupling secondary respiratory therapy device 280 and respiratory therapy device 210B together as shown. Assembly 208 also includes mouthpiece 101A, which includes respiratory therapy sound capturing device 100D. Mouthpiece 101A is coupled to secondary respiratory therapy device 280 via insertion of a portion of mouthpiece 101A into an opening (similar to opening 240) within secondary respiratory therapy device 280 as shown.

FIG. 2K provides a diagram of an assembly 209 of respiratory therapy device 210B, which is coupled to a secondary respiratory therapy device 280 via coupling 282, which extends into secondary respiratory therapy device 280, thereby coupling secondary respiratory therapy device 280 and respiratory therapy device 210B together as shown. Assembly 209 also includes mouthpiece 101C, which includes respiratory therapy sound capturing device 100G. Mouthpiece 101C is coupled to secondary respiratory therapy device 280 via insertion of a portion of mouthpiece 101C into an opening (similar to opening 240) within secondary respiratory therapy device 280 as shown.

FIG. 2L provides a diagram of an assembly 211 of respiratory therapy device 210B, which is coupled to a secondary respiratory therapy device 280 via coupling 282, which extends into secondary respiratory therapy device 280, thereby coupling secondary respiratory therapy device 280 and respiratory therapy device 210B together as shown. Assembly 211 also includes respiratory mask 101B, which includes respiratory therapy sound capturing device 100G. Respiratory mask 101B is coupled to secondary respiratory therapy device 280 via insertion of a portion of coupling 184 into an opening (similar to opening 240) within secondary respiratory therapy device 280 as shown.

As discussed herein, respiratory therapy sound capturing device(s) 100A, 100B, and/or 100C may be permanently or removably coupled to respiratory therapy device 210A and/or 210B and may be configured to detect, measure, and/or record sounds, clicks, taps, gestures, haptics, interface, oscillations, vibrations and/or user interaction with interface 150 made when respiratory therapy device 210A and/or 210B are in use when, for example a patient/user is performing respiratory exercises and/or performing respiratory training, respiratory therapy, diagnostic, and/or a pulmonary function test.

In some embodiments, attachment mechanism(s) 110A and/or 110B may cooperate with a corresponding attachment mechanism(s) positioned on an external surface of respiratory therapy device 210A and/or 210B such as a magnet, track, notch, pin, or snap. In some embodiments (e.g., the embodiments shown in FIGS. 2A and 2B), respiratory therapy sound capturing device(s) 100A, 100B, and/or 100C may be configured to be positioned at a plurality of locations on the exterior surface of respiratory therapy sound capturing device(s) 100A, 100B, and/or 100C via, for example, a track or magnet that accommodates movement and/or repositioning of respiratory therapy sound capturing device(s) 100A, 100B, and/or 100C. In some instances, moving the respiratory therapy sound capturing device(s) 100A, 100B, and/or 100C may be responsive to a quality (e.g., intensity and/or signal to noise ratio (SNR)) and/or frequency of sound that is measured and/or recorded. An initial test measurement or sound recording may be made, processed by controller and/or processor 140 according to, for example, one or more sets of instructions and/or methods disclosed herein that may be recorded on a memory like memory 145, processed by a processor like controller and/or processor 140, and/or communicated to an external device via a port or interface like port 130 and interface 150.

FIG. 3 is a block diagram of an exemplary system 300 configured to execute one or more methods disclosed herein and/or cooperate with one or more respiratory therapy sound capturing devices like respiratory therapy sound capturing device 100A, 100B, and/or 100C to, for example, measure, record, process, and/or analyze data received by the respiratory therapy sound capturing device. System 300 includes one or more respiratory therapy sound capturing devices 100A, 100B, and/or 100C, a user device 305, a respiratory therapy device 210 like respiratory therapy devices 210A and/or 210B, a communication network 310, a computer 315, a cloud computing system 320, a database 325, and a third party system 330. User device 305 may be any personal computing and/or communication device such as a smart phone, tablet computer, or personal computer.

In many instances, communication network 310 is and/or includes the Internet. Additionally, or alternatively, communication network 310 may be a private network (e.g., a LAN) that may be managed by, for example, a health care provider and/or medical treatment facility. The components of system 100 may be directly and/or indirectly communicatively and/or electrically coupled together via one or more wired and/or wireless communication links and/or communication network 310. In some instances, wireless communication of one or more components of system 100 may be enabled using short-range wireless communication protocols designed to communicate over relatively short distances (e.g., BLUETOOTH®, near field communication (NFC), radio-frequency identification (RFID), and Wi-Fi). Exemplary user devices 305 include, but are not limited to, personal electronic devices such as laptop computers, tablet computers, and/or smart phones. The user devices may be configured and/or programmed to execute one or more sets of instructions and/or run one or more software applications to, for example, execute one or more methods disclosed herein.

Cloud computing system 320 may be any cloud computing system configured to receive data from one or more components of system 100 and/or provide data and/or instructions directly and/or indirectly to one or more components of system 100. In some cases, cloud computing system 320 may be configured to run one or more a machine learning programs, train one or algorithms, and/or support a machine learning architecture such as TensorFlow to, for example, perform one or more of the methods disclosed herein. Exemplary cloud computing platforms include, but are not limited to, Amazon Web Service (AWS), Rackspace, and Microsoft Azure. Exemplary machine learning architectures include neural networks, artificial neural networks, Bayesian networks, and/or software or hardware that utilizes artificial intelligence.

Third party system 330 may be any computer or system of computers operated by a third party (e.g., a healthcare provider, a medical professional, and/or medical treatment facility) that may, for example, maintain an electronic medical record for a plurality of patients using respiratory therapy sound capturing device 100A, 100B, and/or 100C and/or a component of system 100. Computer 315 may be configured to, for example, receive data from and/or communicate data and/or instructions to a component of system 100, such as respiratory therapy sound capturing device 100A, 100B, and/or 100C, user device 305 and/or act as a communication terminal to cloud computing system 320 via, for example, communication network 310. Additionally, or alternatively, computer 315 may be configured to facilitate provision of the results of processing received data performed on cloud computing system 320 to display of, for example, computer 315 and/or user device 305. Exemplary computers 315 include desktop and laptop computers, servers, tablet computers, personal electronic devices, mobile devices (e.g., smart phones), and the like. Computer 315, user device 305, third party system 330, and/or cloud computing system 320 may be communicatively coupled to database 325, which may be configured to store received information such a raw data received from respiratory therapy sound capturing device 100A, 100B, and/or 100C, processed data from user device 305, computer 315, and/or cloud computing system 320, and/or one or more sets of instructions that may configured to facilitate execution of one or more processes described herein, or inputs, used for machine learning and/or sets of instructions for user device 305, computer 315, and/or cloud computing system 320 so that, for example, user device 305, computer 315, and/or cloud computing system 320 may execute one or more methods described herein.

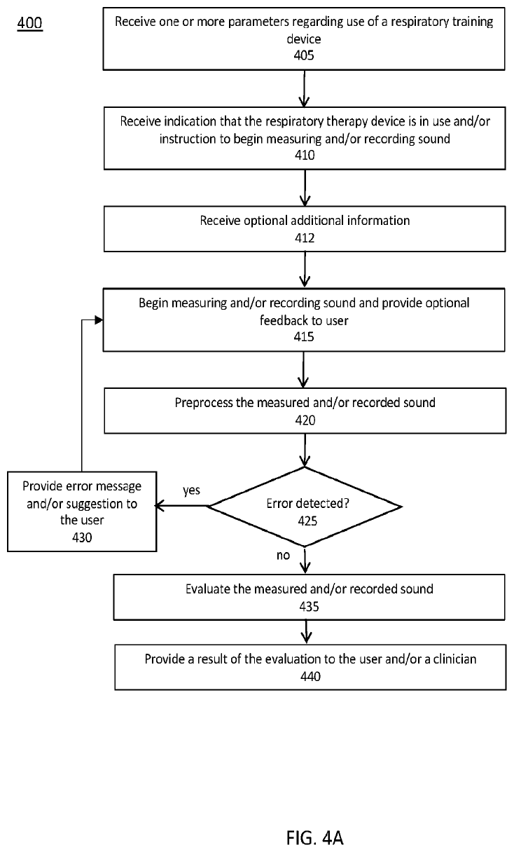

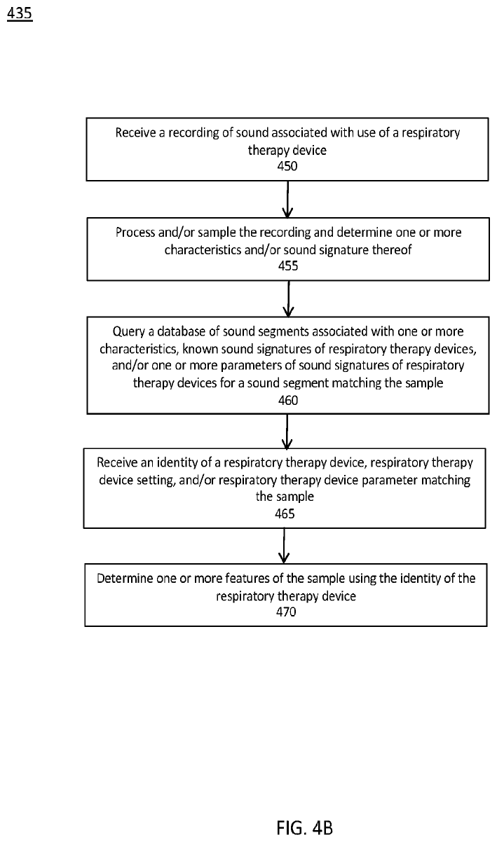

FIG. 4A is a flowchart illustrating a process 400 for using a respiratory therapy sound capturing device such as respiratory therapy sound capturing device(s) 100A, 100B, and/or 100C to measure, record, and/or analyze sound produced during use of a respiratory therapy device such as respiratory therapy device 210A and/or 210B, provide feedback to a user of the respiratory therapy sound capturing device regarding his or her use of the respiratory therapy device, and/or provide an indication of how the user is using the respiratory therapy device to a treatment provider of the user. Process 400 may be executed by any of the systems or system components disclosed herein such as system 300, memory 145, controller and/or processor 140, and/or by a software application communicatively coupled to and/or running on, for example, transceiver 125, controller and/or processor 140, user device 305, computer 310, cloud computing system 320, and/or third party system 330.

Optionally, in step 405, one or more parameters regarding use of a respiratory therapy device for a user (e.g., a respiratory therapy patient and/or user performing respiratory training such as a musician and/or athlete) may be received from, for example, a treatment provider, a respiratory therapist, a respiratory therapy device manufacturer, and/or the user. Parameters for use of the respiratory therapy device may include, but are not limited to, a frequency of use (e.g., twice a day), a time of day (e.g., morning, evening, following exercise, etc.) when the respiratory therapy device is to be used, a prescriptions for use such as a number of repetitions, when to cough or clear mucus from the lungs, a duration of a breath, whether a breath is to be slow and consistently exhaled, holding the breath prior to exhaling or inhaling through the respiratory therapy device, and/or how to orient the respiratory therapy device when in use, and so on. In some embodiments, the parameters received in step 405 may be responsive to one or medical diagnosis of the patient such as asthma, chronic obstructive pulmonary disease (COPD), bronchiectasis, and/or cystic fibrosis. Additionally, or alternatively, the parameters received in step 405 may be responsive to one or medical conditions of and/or medical treatments undergone by the patient such as, a transplant, open heart surgery, removal of a tumor from lung tissue, and/or broken ribs.

In step 410, an indication that a respiratory therapy device is in use and/or may soon be in use may be received. This indication may be, for example, a user turning on a respiratory therapy sound capturing device via, for example, interaction with an interface like interface 150, a user moving the respiratory therapy sound capturing device, thereby initiating a motion-sensitive activation or a respiratory therapy sound capturing device, and/or a detection of a sound generated by the user and/or respiratory therapy sound capturing device. Motion of the respiratory therapy sound capturing device may be detected by, for example, an accelerometer such as accelerometer 135. Sound may be detected by, for example, a microphone such as first microphone 115 and/or second microphone 120.

Additionally, or alternatively, step 410 may include receipt of an instruction to begin measuring and/or recording sound, vibrations, and/or motion of the respiratory therapy device. The instruction may be, for example, received via a user's interaction with an interface like interface 150 and/or a user's interaction with a computer or other electronic device (e.g., user device 305) communicatively coupled to respiratory therapy sound capturing device via, for example, a transceiver like transceiver 125.

In step 415, the respiratory therapy sound capturing device may be measuring and/or recording, for example, sound coming from use of the respiratory therapy device and/or motion of the respiratory therapy device. The recording may be stored on a memory like memory 145 by, for example, a processor and/or communicated to the user device. Optionally, execution of step 415 may include providing feedback to the user regarding his or her use of the respiratory therapy device via, for example, interface 150. The feedback may be, for example, auditory (e.g., a spoken phrase, a click, and/or a beep), visual (e.g., one or more lights blink while the device is in use, a red light blinks when the device is not being used in accordance with one or more parameters, and/or a message is displayed on a display screen of the user interface), and/or audio/visual (e.g., a video showing proper usage of the respiratory therapy device). In some embodiments, the feedback may include one or more prompts directing the user to take one or more actions in a prescribed sequence. For example, when the respiratory therapy device is a medication inhaler, a red light may go on when movement of the inhaler is detected and may stay on until the user shakes the inhaler for a period of time (e.g., 5 s) included in the parameters received in step 405. Once the sound corresponding to shaking of the inhaler has been received for 5 seconds, the red light may go off and the green light may go on, thereby prompting the user to stop shaking the inhaler and initiate step two of the medication inhalation process (e.g., exhaling). Once sound indicating user exhalation is detected, the green light may go off, the red light may come back on, and may stay on until the exhalation is finished. Once the exhalation and sound indicative of an inhalation is detected, the green light may go on, thereby prompting the user to inhale medication from the inhaler into his or her lungs.

Optionally, in step 420, the measured and/or recorded sound may be pre-processed to, for example, remove noise and/or undesired frequencies of sound, and/or amplify preferred frequencies of sound. In some instances, noise reduction in the recording may be performed using information regarding ambient sound that may be detected by, for example, a microphone such as first or second microphone 115 and/or 120. In step 425, the measured and/or recorded sound of step 415 or 420 may be analyzed to determine if an error is detected and, if so, an error message and/or a suggestion for adjusting a technique of using the respiratory therapy device may be provided to the user (step 430). Exemplary messages that may be provided in step 430 include, but are not limited to, “shake, shake, shake,” “inhale, inhale, inhale,” or “exhale slowly and steadily” Errors detected in step 425 may include errors detected with regard to an operation of the respiratory therapy device and/or a failure of the respiratory therapy sound capturing device to properly receive and/or measure the sound and/or motion produced by the respiratory therapy device, which may be caused by, for example, a faulty mechanical coupling of the respiratory therapy device to the respiratory therapy device and/or user error.

When the sound measured and/or recorded is not subject to an error, it may be evaluated to determine one or more characteristics thereof and/or whether the user's use of the respiratory therapy device is compliant with one or more of the parameters received in step 405 (step 435) and a result of the evaluation may be provide to the user and/or a clinician or medical treatment provider in step 440 via, for example, a computer display device and/or interface 150. Exemplary characteristics include, but are not limited to, how long the respiratory therapy device is used, how many breaths the user exhaled and/or inhaled into the respiratory therapy device, a duration of breaths, and/or whether the user coughed during and/or following use of the respiratory therapy device. For example, first and/or second microphone 115 and/or 120 may be used to record sound made during the user's inhalation to determine whether it is of sufficient length and/or is too loud (which may be an indication that the inhalation is too large and/or above a parameter for recommended inhalation air flow rate and/or volume). If a user's inhalation is determined to be non-compliant with one or more inhalation parameters, a message indicating same may be provided to the user in step 440 and/or a suggestion for how to take a more compliant inhalation may be provided to the user via, for example, interface 150 and/or a message delivered to his or her user device. Additionally, or alternatively, first and/or second microphone 115 and/or 120 may be used to record sound made during the user's use of the respiratory therapy device and it may be evaluated in step 435 to determine when and/or whether the user is exhibiting any signs of respiratory distress (e.g., a high number of shallow breaths, wheezing, coughing, etc.) and/or a trend consistent of a worsening and/or an exacerbation of a respiratory condition that may need an intervention and/or a change in one or more parameters of step 405 may be desired and/or necessary. This determination may then be compared with a parameter received in step 405 to determine, for example, if the user is using the respiratory therapy device in a manner that is compliant with the parameter and/or whether the user is experiencing respiratory distress. An indication of the determination of step 435 may be provided in step 440 to, for example, the user and/or a clinician or other medical professional via, for example, the user device, a computer, and/or a third party. Additionally, or alternatively, an error flag may be associated with all and/or a portion of sound recorded via a particular execution of process 400 that may, for example, indicate that the recorded sound and/or a portion thereof should be removed from a sample set of sound recorded for the user.

Optionally, in some embodiments, (e.g., when the evaluation indicates a need to evaluate a patient's breathing or lung function) the result provided to the user in step 440 may include an instruction to obtain additional physiological measurements such as an instruction to remove respiratory therapy sound capturing device(s) 100A, 100B, and/or 100C from respiratory therapy device 210A and attach it to a device that may be configured to perform auscultation in a manner similar to a stethoscope device. Then, the user may be instructed to hold respiratory therapy sound capturing device(s) 100A, 100B, and/or 100C that may be attached to the stethoscope device against their chest or back for a period of time and/or number (e.g., 3-10) deep or shallow breaths so that first and/or second microphone 115 and/or 120 may detect and/or record sound produced by the user's lungs and/or chest while he or she is breathing. Alternatively, the instruction may be to use an alternative device (e.g., a peak flow meter and/or spirometry device) to collect one or more measurements of pulmonary function.

Exemplary data that may be received in step 412, measured and/or recorded in step 415, and/or a result of an evaluation of step 435 may include, but is not limited to, a time stamp, session duration time, geolocation of the respiratory therapy sound capturing device providing the measurements and/or recordings, a number of inhalations and/or exhalations during a period of time, an average inhalation and/or exhalation duration, a peak inhalation and/or exhalation duration, inspiratory flow rate, expiratory flow rate, a peak inspiratory and/or expiratory amplitude, a total inspiratory and/or expiratory volume, trends in values determined over time (e.g., weeks, months, years), and/or usage information such as a number of times per day, week, and/or month the user has used the respiratory therapy device. In some occasions, an evaluation determined in step 435 may include evaluation of how closely the user complied with one or more parameters (received in, for example, step 405) while using the respiratory therapy device. On these occasions, a respiratory therapy device score may be determined in step 435 and provided to the user and/or clinician in step 440.

In one embodiment, the respiratory therapy sound capturing device(s) disclosed herein may be configured as part of a modular system that is configured to collect different types of acoustic data (e.g., use of the respiratory therapy device, background noise, noise generated by the user's lungs while breathing, noise generated by the user while coughing, time and/or location of recording, etc.) via one or more microphones as well as motion data that may be used to determine how ambulatory the user is and/or how much he or she is moving while using the respiratory therapy device.

On some occasions, the user may wear the respiratory therapy sound capturing device(s) 100A, 100B, and/or 100C for all, or a portion (e.g., waking hours) of a day or set of continuous days to monitor various sounds (coughs, snores, rapid breathing, wheezing, silence, gaps in breathing (as may happen with sleep apnea), etc.) and/or motion of the user. Wearing of respiratory therapy sound capturing device(s) 100A, 100B, and/or 100C may be facilitated by, for example, a bracelet, strap, or necklace that respiratory therapy sound capturing device(s) 100A, 100B, and/or 100C is permanently or removably attached to. At times, respiratory therapy sound capturing device(s) 100A, 100B, and/or 100C may be configured to use a wired and/or wireless charging station to charge a battery of power source 155.

In some instances, respiratory therapy sound capturing device(s) 100A, 100B, and/or 100C may be configured to cooperate with a software application running on a user device (e.g., smart phone or computer) and/or a server via communication facilitated by transceiver 125 over a short-range communication protocol and/or communication with the Internet and/or a cloud-based computing environment.

In one use case, a user may couple respiratory therapy sound capturing device(s) 100A, 100B, and/or 100C to a battery charging device prior to going to sleep so that it may charge overnight. Upon waking, the user may decouple the battery charging device from respiratory therapy sound capturing device(s) 100A, 100B, and/or 100C and then wear it via, for example, attaching it to a bracelet or necklace. Respiratory therapy sound capturing device(s) 100A, 100B, and/or 100C may be programmed with a schedule for use of the respiratory therapy device and may remind the user throughout the day to use his or her respiratory therapy device in a prescribed manner. At that point, the user may mechanically couple respiratory therapy sound capturing device(s) 100A, 100B, and/or 100C to the respiratory therapy device in, for example, a manner like that described above with regard to FIG. 2A. Then, process 400 or portions thereof may be executed, and, upon completion, the user may decouple respiratory therapy sound capturing device(s) 100A, 100B, and/or 100C from the respiratory therapy device and resume wearing it.

In one use case, the respiratory therapy device used by the user may be an inhaler and parameters for its use that are received in step 405 may include a requirement that 1) the user shake the inhaler for 5 seconds prior to use, 2) the user exhale and then begin inhaling, and 3) the user activate dispensing of medication to the user via mouth as the user is inhaling. Then, in step 410, an indication that the respiratory therapy device is in use may be received via, for example, detected motion of a respiratory therapy sound capturing device coupled to the respiratory therapy device and the respiratory therapy sound capturing device may be measuring and/or recording sound (step 420), which may be evaluated (step 435) to determine, for example, 1) whether the user shook the inhaler and, if so, for how long, 2) whether sound corresponding to the user's exhalation and an initial portion of an inhalation are recorded and/or measured, and/or 3) whether sound corresponding to a user's inhalation of medication dispensed by the inhaler into the patient's mouth may be heard and a result of the evaluation may be provided to, for example, the user to, for example, train the user in the use of the inhaler and monitor the user's use of the inhaler and/or determine whether the user is using the inhaler correctly and/or in accordance with one or more of the parameters received in step 405.

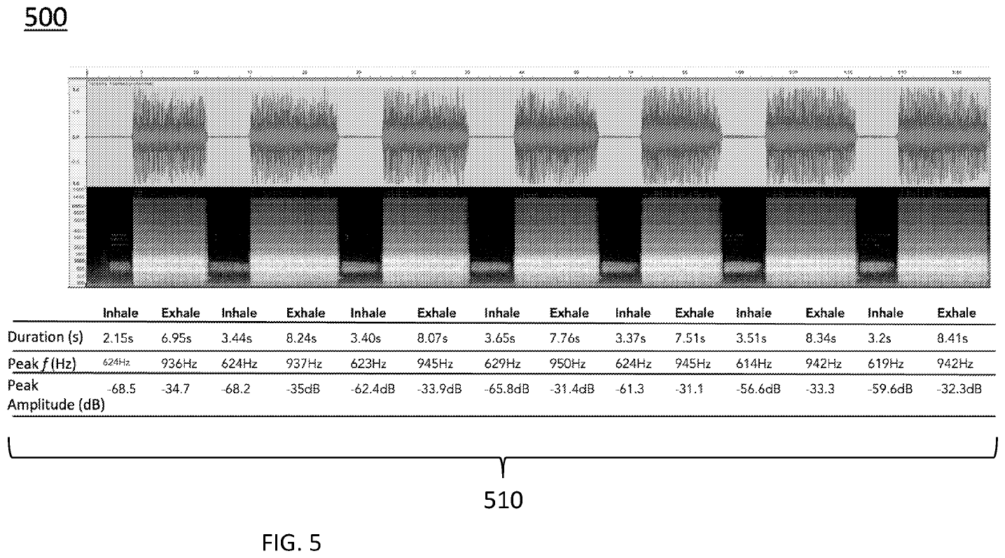

In another use case, the respiratory therapy device may be a PEP device, an OPEP device, a RMT device, and/or incentive spirometer device and the parameters for its use that may be received in step 405 may include, for example, a number of exhalations to breathe into the OPEP device, a target duration for a single exhalation into the OPEP device, and/or a time period for how long to use the OPEP device each day and/or each session. FIG. 5 provides a spectrogram 500 showing sound recorded by a respiratory therapy sound capturing device like respiratory therapy sound capturing device 100A, 100B, and/or 100C while a user is using a respiratory therapy device in the form of a OPEP device as part of, for example, execution of process 400. Spectrogram 500 corresponds to sound recorded for 1 minute and 23 seconds and captures sounds made as the user inhales and exhales seven times with an average duration for inhalation being 3.24 seconds and an average duration of exhalation being 7.89 seconds. Specific data for each inhalation and exhalation shown in spectrogram 500 is provided in table 510, which provides a duration, peak frequency, and peak amplitude for each inhalation and exhalation. This data may be analyzed to determine, for example, whether the user is using the OPEP device according to one or more parameters for its use and/or whether the user's respiratory condition is improving and/or declining when comparing the results of spectrogram 500/table 510 with other prior-recorded results.

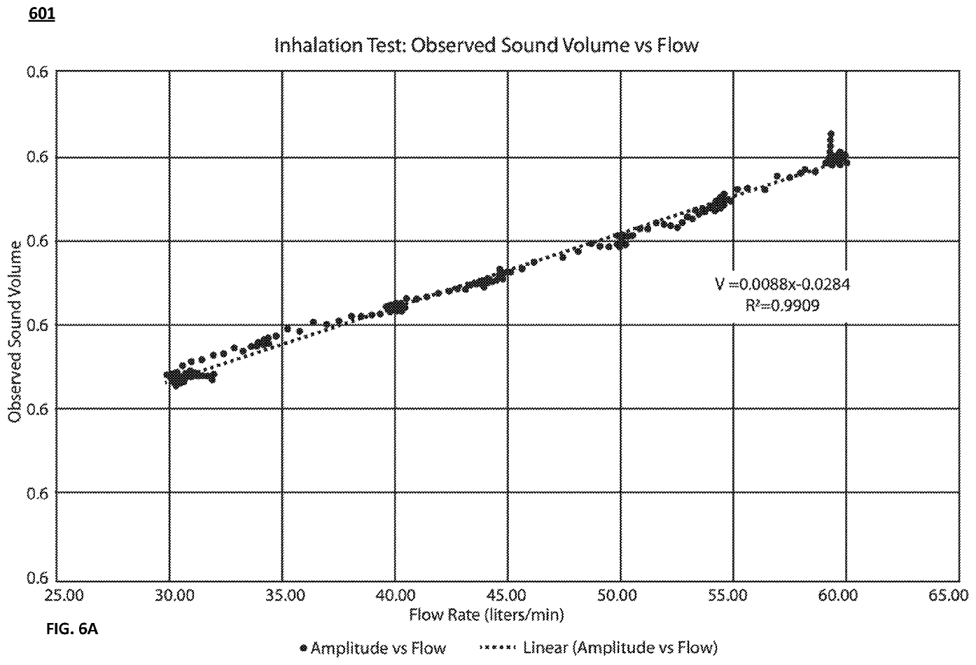

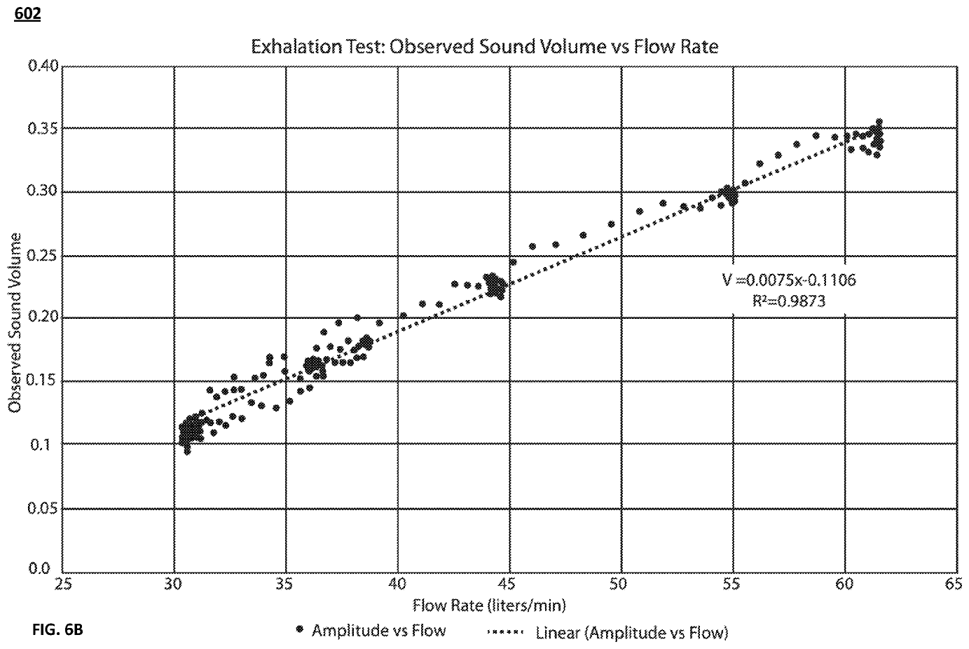

In some embodiments, evaluation results generated via execution of step 435 may be provided in graph form. For example, FIG. 6A provides a graph 601 showing values (as dots) for observed sound volume, or intensity, measured in decibels as a function of air flow rate measured in liters per minute for a particular user using a system including a respiratory therapy device (e.g., respiratory therapy device 210B) that is coupled to one or more of the respiratory therapy sound capturing device(s) disclosed herein when the user in inhaling through a system that includes a respiratory therapy device (e.g., respiratory therapy device 210B) and a respiratory therapy sound capturing device as disclosed herein. Graph 601 also shows a linear regression line that relates amplitude as a function of flow rate when the user is inhaling.

FIG. 6B provides a graph 602 showing values (as dots) of observed sound volume, or intensity, measured in decibels as a function of air flow rate measured in liters per minute for a particular user using a system including a respiratory therapy device (e.g., respiratory therapy device 210B) that is coupled to one or more of the respiratory therapy sound capturing device(s) disclosed herein when the user in exhaling through a system that includes a respiratory therapy device (e.g., respiratory therapy device 210B) and a respiratory therapy sound capturing device as disclosed herein. Graph 602 also shows a linear regression line that relates amplitude as a function of flow rate when the user is exhaling.

The information from graphs 601 and/or 602 and/or the linear regression formulas derived therefrom may be used to make predictions and/or calculate a user's inspiratory or expiratory flow rate(s) using, for example, a sound volume observed when a user is inhaling or exhaling through a system including a respiratory therapy device (e.g., respiratory therapy device 210B) that is coupled to one or more of the respiratory therapy sound capturing device(s) disclosed herein. In some embodiments, information like the information depicted on graphs 601 and/or 602 and/or the information derived therefrom may be specific to a particular type of respiratory therapy device and/or respiratory therapy sound capturing device being used and, in these embodiments, information regarding the respiratory therapy device and/or the respiratory therapy sound capturing device being used may be received in, for example, step 412, and/or may be associated with a recording of sound made by the respiratory therapy sound capturing device.

In some embodiments, execution of step 435 may include performing one or more fast Fourier transform (FFT) with and/or on one or more sound recordings received from one or more of the respiratory therapy sound capturing device(s) disclosed herein, which may result in evaluating and/or determining of an average amplitude of sound received for one or more frequencies included in a recording.

FIG. 4B is a flowchart illustrating an exemplary process for executing step 435 to evaluate a sample of a recording of sound associated with use of a respiratory therapy device, that may be executed by any of the systems or system components disclosed herein.

Initially, in step 450, a processor or computing device may receive a recording of sound associated with use of a respiratory therapy device (such as the respiratory therapy devices disclosed herein) from, for example, one or more respiratory therapy sound capturing devices, such as the respiratory therapy sound capturing devices disclosed herein. The recording may be processed and/or sampled to determine one or more characteristics thereof (step 455). Exemplary processing may include, but is not limited to, filtering the recording to remove ambient noise, amplifying one or more frequencies of sound, and/or performing a FFT on the recording to, for example, determine sound amplitude as function of frequency that is captured in the received recording. Exemplary sampling performed in step 455 may include extracting a portion of the recording (e.g., 0-2.0 s; 0-5.0 s; 5.0-10.0 s, etc.) to determine one or more characteristics thereof. Exemplary characteristics include, but are not limited to, a set of frequencies and corresponding amplitudes for sound included in the sample, a level of noise and/or interference in the sample, and/or frequencies included in the sample, which may collectively and/or individually be referred to herein as a “sound signature”.

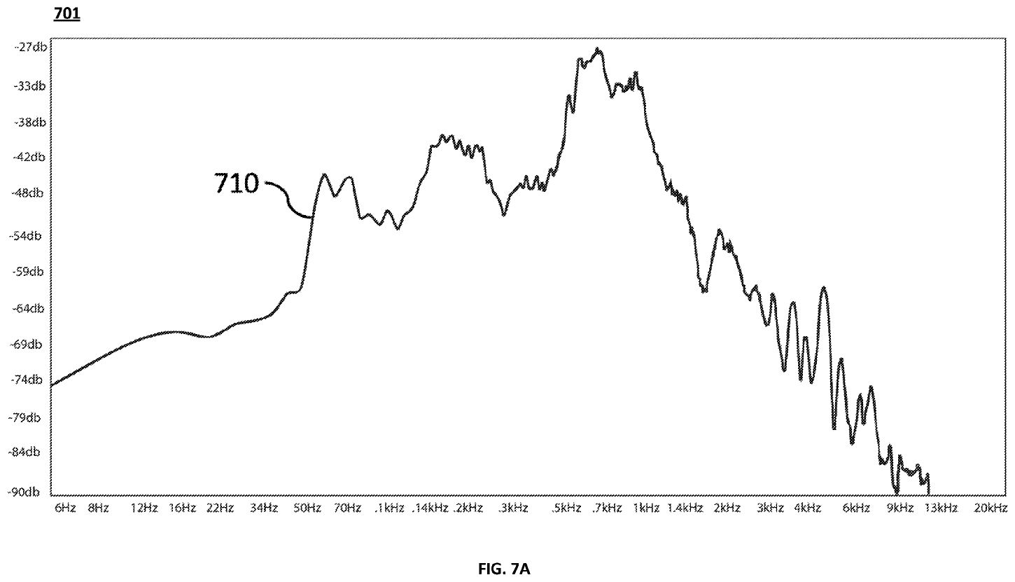

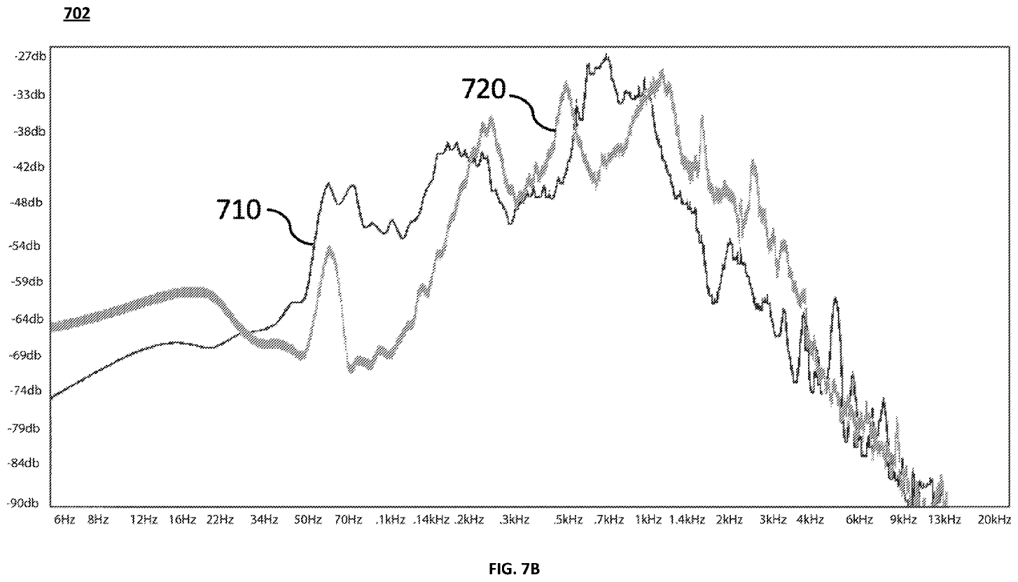

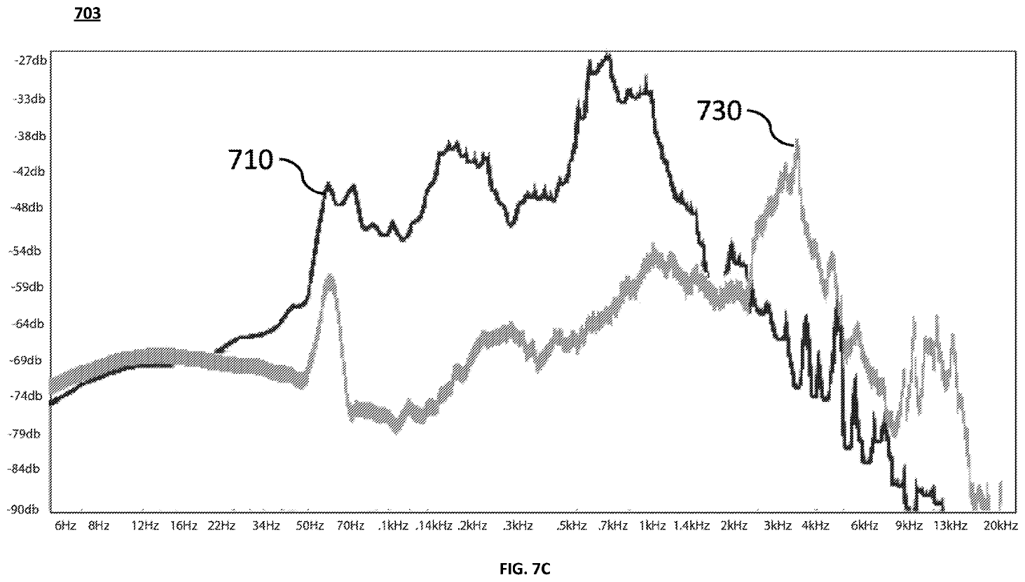

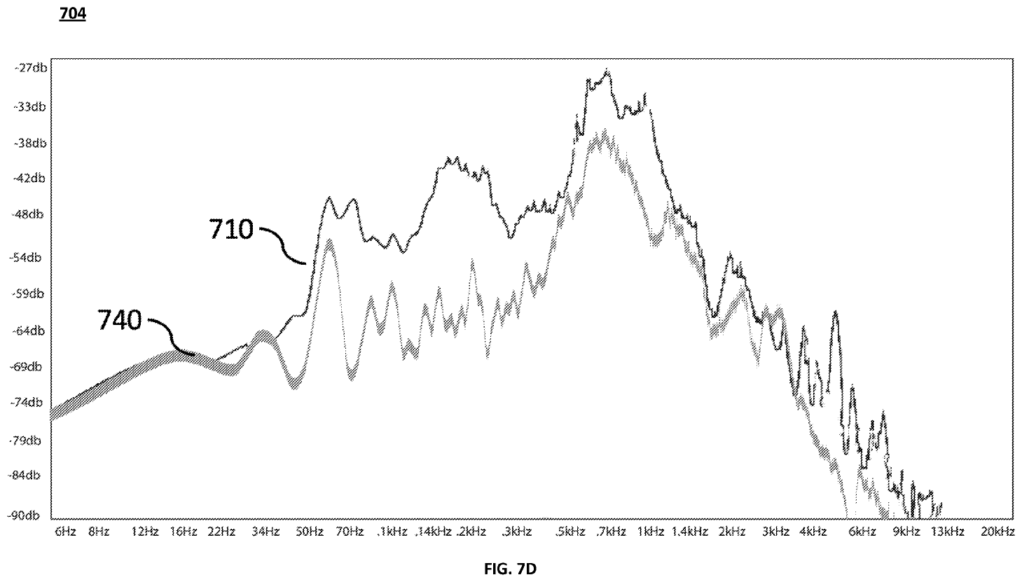

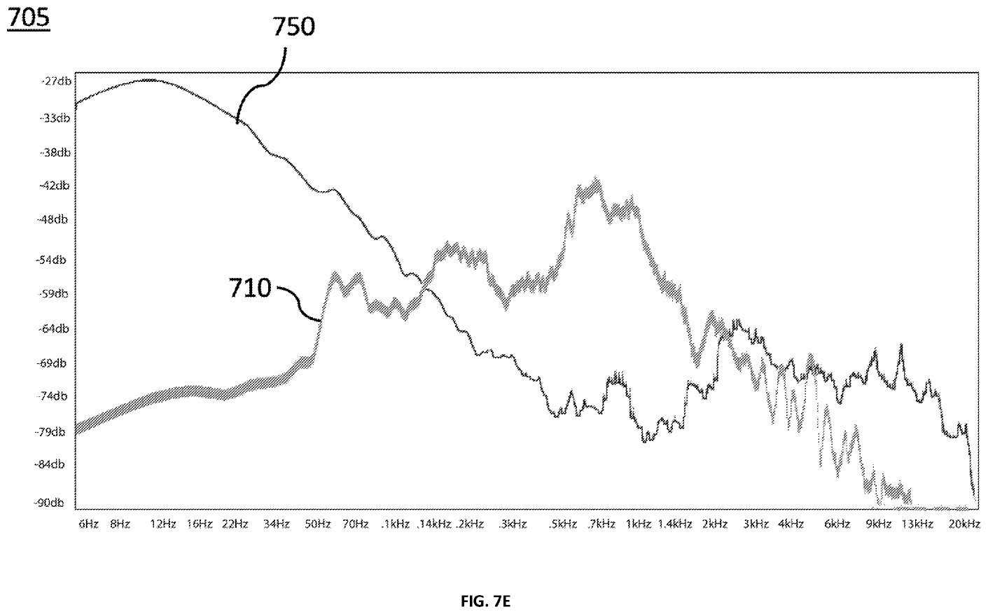

FIG. 7A provides a graph showing a first relationship 710 (in the form of a line) between sound amplitude (measured in decibels) as a function of frequency (measured in Hz) for a sample of a recording made when a user is using a first respiratory therapy device. First relationship 710 may be determined via execution of step 455 and may represent a sound signature of the first respiratory therapy device, which may be saved as a known sound signature associated with a user is using the first respiratory therapy device in a database that is later queried in, for example, step 460.

In step 460, a database (e.g., database 325) of sound segments associated with one or more characteristics, known sound signatures of respiratory therapy devices, and/or one or more parameters of sound signatures of respiratory therapy devices may be queried for a sound segment matching a sound signature and/or characteristic of the recording and/or sample. In step 465, an identity of a respiratory therapy device, respiratory therapy device setting, and/or parameter that matches the query may be received from the database and used to determine one or more features (e.g., duration, number of inhales, number of exhales, etc.) of the sample.

FIG. 7B provides a graph 702 showing relationship 710 (which is a known sound signature associated with a user is using the first respiratory therapy device) overlaid upon a second relationship 720 (in the form of a line) between sound amplitude (measured in decibels) as a function of frequency (measured in Hz) for a sample of a recording that may be received in, for example, step 450 and/or sampled in step 455 when a user is using a second, or unknown, respiratory therapy device. Comparison of the first and second relationships (as may be done via, for example, execution of step(s) 455-465) shows how the first and second respiratory therapy devices differ from one another with regard to a sound signature made by the respective first and second respiratory therapy devices. Thus, if a sample with a sound signature similar to second relationship 720 is received and/or determined in steps 450 and/or 455, it may be used to query the database in step 460 to determine that the use is not using the first respiratory device and/or find a respiratory therapy device (in this case, the second respiratory device) that matches a sound signature for a respiratory device the user is using (step 465).

Additionally, or alternatively, FIG. 7C provides a graph 703 showing relationship 710 (which is a known sound signature associated with a user is using the first respiratory therapy device) overlaid upon a third relationship 730 (in the form of a line) between sound amplitude (measured in decibels) as a function of frequency (measured in Hz) when a user is using a third respiratory therapy device. Comparison of the first and third relationships (as may be done via, for example, execution of step(s) 455-465) shows how the first and third respiratory therapy devices differ from one another with regard to a sound signature made by the respective first and third respiratory therapy devices. This information may be used to, for example, indicate that the user is not using the first respiratory therapy device and/or is using the third respiratory therapy device in step 465.

Additionally, or alternatively, FIG. 7D provides a graph 704 showing relationship 710 (which is a known sound signature associated with a user is using the first respiratory therapy device at a first setting (e.g., level of resistance)) overlaid upon a fourth relationship 740 (in the form of a line) between sound amplitude (measured in decibels) as a function of frequency (measured in Hz) when a user is using the first respiratory therapy device but with a different setting during a sample received in step 450 and/or generated in step 455. Comparison of the first and fourth relationships shows how different settings of the first respiratory therapy devices differ from one another and this setting of the first respiratory devices for the sample of fourth relationship 740 may be received in step 465 and/or provided to the user in step 440.

In some embodiments, execution of step 435 may include looking for a sound signature of a sound corresponding to an action that involves, for example, preparing the respiratory therapy device for use (e.g., assembly or shaking). For example, FIG. 7E provides a graph 705 showing relationship 710 overlaid upon a fifth relationship 750 (in the form of a line) between sound amplitude (measured in decibels) as a function of frequency (measured in Hz) when a user is shaking the respiratory therapy device and/or respiratory therapy sound capturing device extracted from a sound recorded received in step 450. When step 460 is executed using a sample represented by fifth relationship 750, an indication that the user is shaking the respiratory therapy device may be received in step 465 and/or a feature of the shaking (e.g., duration) may be determined in step 470.

The comparisons shown in graphs 701, 702, 703, 704, and 705 may be used to, for example, determine which respiratory therapy device a user is using, which setting a respiratory therapy device may be set to during use, and/or what the user is doing (e.g., inhaling, exhaling, or shaking) during a duration over which sound is being recorded.

In some embodiments, the devices, systems, and assemblies disclosed herein may set a known distance between a microphone that is detecting, measuring, and/or recording sound and a source of the sound (e.g., sound-producing device 194, mouthpiece 101A and/or 101C, and/or respiratory mask 101B), which may be useful when analyzing and/or evaluating one or more recordings of sound made with one or more of the respiratory therapy sound capturing device disclosed herein. For example, because sound intensity is known to follow the Inverse Square Law (i.e., intensity is proportional to the inverse of the distance squared), an intensity for sound recorded by one or more of the respiratory therapy sound capturing devices disclosed herein may be calculated using the distance of the microphone recording the sound from the source of the sound.

I claim:

1. A respiratory therapy sound capturing device comprising:
an attachment mechanism configured to attach the respiratory therapy sound capturing device to a respiratory therapy device;
a microphone configured to detect sound made when a user is using the respiratory therapy device and communicate the detected sound to a transceiver; and
the transceiver, the transceiver being communicatively coupled to the microphone and transmit sound detected by the microphone to an external computing device.

2. The respiratory therapy sound capturing device of claim 1, further comprising:
an accelerometer configured to detect at least one of motion of the respiratory therapy sound capturing device and an orientation of the respiratory therapy sound capturing device.

3. The respiratory therapy sound capturing device of claim 1 or 2, wherein the respiratory therapy device is at least one of an inhaler, a positive expiratory pressure (PEP) device, oscillating positive expiratory pressure (oPEP) device, incentive spirometer, respiratory muscle trainer (RMT), spirometer, nebulizer, and a peak flow meter.

4. The respiratory therapy sound capturing device of any of claims 1-3, wherein the attachment mechanism is an adhesive.

5. The respiratory therapy sound capturing device of any of claims 1-4, wherein the respiratory therapy device includes a mouthpiece and a body and the respiratory therapy sound capturing device is configured to be placed between the mouthpiece and the body of the respiratory therapy device to capture sound the user makes when inhaling out of the mouthpiece or exhaling into the mouthpiece.

6. The respiratory therapy sound capturing device of claim 5, wherein the respiratory therapy sound capturing device further comprises:
a lumen configured to allow air exhaled into mouthpiece to be communicated to the body of the respiratory therapy device and is also configured to allow air inhaled through the body of the respiratory therapy device to be communicated to the mouthpiece.

7. The respiratory therapy sound capturing device of any of claims 1-6, wherein the sound detected by the microphone is sound corresponding to at least one of air moving into the respiratory therapy device, air moving out of the respiratory therapy device, a vibration of the respiratory therapy device, and an oscillation of a component air of the respiratory therapy device.

8. A system comprising:
a respiratory therapy device; and
a respiratory therapy sound capturing device coupled to the respiratory therapy device, the respiratory therapy sound capturing device comprising:
a microphone configured to detect sound made when a user when the user is using the respiratory therapy device and communicate the detected sound to a transceiver; and
the transceiver, the transceiver being communicatively coupled to the microphone and transmit sound detected by the microphone to an external computing device.

9. The system of claim 8, wherein the respiratory therapy sound capturing device further comprises:
an accelerometer configured to detect at least one of motion of the respiratory therapy sound capturing device and an orientation of the respiratory therapy sound capturing device.

10. The system of any of claims 8 or 9, wherein the respiratory therapy device is at least one of an inhaler, a positive expiratory pressure (PEP) device, oscillating positive expiratory pressure (oPEP) device, a peak flow meter, Incentive spirometer, and a respiratory muscle trainer device.

11. The system of any of claims 8-10, wherein the respiratory therapy device includes a mouthpiece and a body and the respiratory therapy sound capturing device is configured to be placed between the mouthpiece and the body of the respiratory therapy device to capture sound the user makes when inhaling out of the mouthpiece or exhaling into the mouthpiece.

12. The system of claim 11, wherein the respiratory therapy sound capturing device further comprises:
a lumen configured to allow air exhaled into mouthpiece to be communicated to the body of the respiratory therapy device and is also configured to allow air inhaled through the body of the respiratory therapy device to be communicated to the mouthpiece.

13. The system of any of claims 8-12, wherein the sound detected by the microphone is sound corresponding to at least one of air moving into the respiratory therapy device, air moving out of the respiratory therapy device, a vibration of the respiratory therapy device, and an oscillation of a component of the respiratory therapy device.

14. A method comprising:
receiving, by a processor, a parameter for using a respiratory therapy device;
receiving, by the processor, sound detected by a microphone when a user when the user is using the respiratory therapy device and communicate the detected sound to a transceiver;
analyzing, by the processor, the received sound to determine if the received sound corresponds to use of the respiratory therapy device in a manner that is compliant with the received parameter; and
providing a result of the analysis to a display device.

15. The method of claim 14, wherein the analysis includes determining at least one of a number of inhales in a time period, a number of exhales in a time period, a duration of inhales in a time period, and a duration of exhales in a time period.

16. The method of claim 14 or 15, wherein the analysis includes determining at least one of a peak frequency and a peak amplitude detected for one or more exhalations detected during a time period.

17. The method of any of claims 14-16, wherein the analysis includes determining at least one of an average frequency, a peak frequency, an average amplitude, a peak amplitude, and a duration detected for one or more inhalations detected during a time period.

18. The method of any of claims 14-17, further comprising:
determining, by the processor, a recommendation regarding how to use the respiratory therapy device responsively to the analysis; and
providing, by the processor, the recommendation to the user.
